(19) United States
(12) Patent Application Publication
(10) Pub. No.: US 2025/0000485 A1
(43) Pub. Date: Jan. 2, 2025

(54) HYDROGEL COUPLANT SLEEVE FOR USE WITH INTRAORAL ULTRASONIC IMAGING PROBE

(71) Applicant: CARESTREAM DENTAL LLC, Atlanta, GA (US)

(72) Inventors: Seshadri JAGANNATHAN, Rochester, NY (US); Lawrence FOLTS, Macedon, NY (US); Arnaud CAPRI, Paris (FR); Stephanie CHEVALLIOT, Paris (FR)

(21) Appl. No.: 18/690,447

(22) PCT Filed: Sep. 21, 2022
(86) PCT No.: PCT/US22/44238

§ 371 Date: Mar. 8, 2024

Related U.S. Application Data
(60) Provisional application No. 63/246,838, filed on Sep. 22, 2021.

(51) Int. Cl. A61B 8/00

(52) U.S. Cl. CPC A61B 8/4281; A61B 8/4444

(57) ABSTRACT

A hydrogel couplant system for use in ultrasonic imaging includes a sleeve body comprising a hydrogel couplant material; wherein the sleeve body is configured to encapsulate an ultrasonic probe and overcome an impedance mismatch between air and an object being imaged. In one embodiment, the hydrogel couplant material is integrally formed within the body of the sleeve body. In another embodiment, the hydrogel couplant material is adhered to either substantially all of, or a portion of, the outer surface of the sleeve body.

### TECHNICAL FIELD

This application relates to a hydrogel ultrasound couplant sleeve for use with an intraoral imaging probe.

### BACKGROUND

Even though ultrasonic imaging is commonly used in medical imaging, use of this imaging technique presents unique challenges when applied to dental imaging, especially intraoral imaging. A primary reason for these challenges, is that ultrasonic imaging, unlike optical or x-ray imaging, requires the use of a coupling agent (couplant) to overcome the impedance mismatch between air and the object being imaged (human tissue in the case of medical imaging or dental imaging). The function of the couplant is to eliminate the presence of air in the path of transmittance between the ultrasonic probe and the object being imaged.

Typically, the couplants used for this purpose are hydrogels (which are defined by Merriam Webster dictionary as “a gel composed usually of one or more polymers suspended in water”), which match the ultrasonic, physical, chemical and biological properties of human tissues that are being imaged. In the absence of such a couplant, most of the ultrasonic energy would be reflected at the air/tissue interface, making it difficult, if not impossible to image the tissue.

In the case of the medical ultrasonic imaging, the hydrogel couplants are coated on the surface of the probe, and then the probe is placed on the skin surface of the patient. However, in the case of intraoral dental imaging, this approach is unsatisfactory, as the presence of saliva in the mouth results in the dissolution of the hydrogel and removes it from the surface of the ultrasonic probe. This approach has also been considered in intraoral therapeutic applications but suffers from the same drawbacks.

An alternative approach used in therapeutic ultrasound is the use of a hydrogel pad. But this method also presents challenges, which have to be overcome in order for it to be of value in intraoral applications. Specifically, in the case of intraoral imaging, the probe has to be moved across a defined region, so as to image the entire area, and the couplant would have to be continuously moved and repositioned, as the entire gingiva line is imaged. This would make it very difficult, if not impossible to achieve the necessary image quality, and the patient would be subjected to extended periods of discomfort.

Another technique is to pump water into the gap between the ultrasound probe and the gingiva surface, but it is difficult to maintain a constant volume of water in a patient's mouth, without causing appreciable discomfort for the patient.

As a result, the use of ultrasonic imaging as a imaging tool in intraoral dentistry has been severely inhibited. Hence, there is an urgent need for the development of ultrasound couplant system that resolves all the issues discussed above, for use in intraoral imaging, in general, and in periodontal imaging in particular.

### SUMMARY

A hydrogel couplant system for use in ultrasonic imaging includes a sleeve body comprising a hydrogel couplant material; wherein the sleeve body is configured to encapsulate an ultrasonic probe and overcome an impedance mismatch between air and an object being imaged. In one embodiment, the hydrogel couplant material is integrally formed within the body of the sleeve body. In another embodiment, the hydrogel couplant material is adhered to either substantially all of, or a portion of, the outer surface of the sleeve body, such that it covers at least the acoustic windows of the ultrasonic probe. In yet another embodiment, the hydrogel couplant material is bonded directly to the ultrasonic probe.

### BRIEF DESCRIPTION OF THE DRAWINGS

FIG. 1 is a schematic cross-sectional representation of a sleeve body that has been substantially covered with a hydrogel couplant material.

FIG. 2 is a schematic cross-sectional representation of a sleeve body that has been partially covered with a hydrogel couplant material.

FIG. 3A is a schematic cross-sectional representation of a sleeve body in which the hydrogel couplant material has been integrally formed with the sleeve body.

FIG. 3B is a schematic cross-sectional representation of an ultrasonic probe, in which the hydrogel couplant covering is limited to the acoustic windows.

FIG. 4 is a side view of one embodiment of an ultrasonic probe.

FIG. 5A is a cross-sectional view of one embodiment of a sleeve body configured to be disposed over an ultrasonic probe.

FIG. 5B is a side view of one embodiment of a sleeve body configured to be disposed over an ultrasonic probe.

FIGS. 6A and 6B are side views of another embodiment of a sleeve body configured to be disposed over an ultrasonic probe.

FIG. 7 is a side view of one embodiment of a sleeve body disposed over an ultrasonic probe.

FIG. 8 is a perspective view of one example of a sleeve body incorporating a hydrogel couplant material.

### DETAILED DESCRIPTION

A couplant sleeve 10 for use with an intraoral ultrasonic probe 12 is provided. As discussed above, an ultrasonic probe or tool may be used for intraoral imaging to capture ultrasound images within a patient's mouth and used in the dental diagnostic process. While the probe 12 will not be discussed in detail, it should be understood that any probe capable of fitting inside a user's mouth and capturing relevant ultrasound images may be used in combination with the sleeve 10. Similarly, the sleeve 10 may be formed into various shapes corresponding and conforming to the outer dimensions of the probe 12, the sleeve 10 at least covering the probe's acoustic window(s) (i.e., the outer surface(s) of the probe from which the ultrasonic waves are emitted and/or received). Further, the sleeve 10 is designed to move with the probe 12 during the course of an intraoral imaging process without losing intimate contact with the surfaces of the patient's gingiva. Moreover, the sleeve 10 is designed to withstand tearing and maintain properties necessary for ultrasound transmittance. The sleeve 10 is designed to be customized with respect to taste (i.e. the flavor perceived by the patient) and hypoallergenic, antiviral, and antibacterial properties.

The couplant sleeve 10 may include a suitable hydrogel couplant material. The material may be integrally formed within the sleeve 10, as shown in FIG. 3A. In this embodiment, the sleeve 10 is designed to embed the hydrogel couplant material within a suitable polymer. The sleeve is generally composed of a polymeric material, or combination of materials, that is compatible with surface of a patient's gingiva or any other organ that is under ultrasonic examination and shaped to provide additional thickness, if needed, at probe imaging windows (not shown). This embodiment enables the coupling functional to remain active during the entire imaging process. Thus, it limits the number of interruptions during the exam and minimizes discomfort to the patient. In addition, it maintains the overall image quality by limiting the number of acoustic barriers along the ultrasonic waves path.

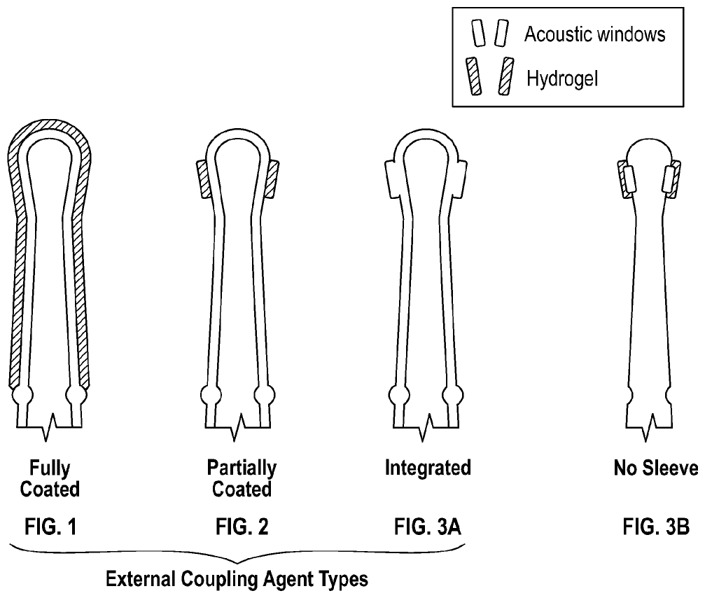

In another embodiment, the hydrogel couplant material 14 may be deposited or coated on to the sleeve 10. The hydrogel couplant material may cover all, or substantially all, of the sleeve's 10 outer surface (FIG. 1) or may be coated on to a small portion of the sleeve 10 (FIG. 2). In this embodiment, the sleeve 10 may be made from a polyethylene material, however it should be understood that the sleeve may be made of any material that can serve as a barrier layer to prevent the transport of moisture and/or saliva, with any dissolved or dispersed moieties from a patient's mouth to the ultrasonic probe. The hydrogel couplant material 14 may be adhered to the outer surface of the sleeve 10 using any suitable method.

Once formed, the sleeves 10 may be sealed in clean or sterilized, single use packages that are easy to open. Once removed from its packaging, the sleeve 10 can be easily positioned onto the probe 12 and, if necessary, can be moisturized by dipping the sleeve 10 in a cup of water or under a running water supply prior to or subsequent to attachment to the probe 12.

In yet another embodiment, the hydrogel may be applied directly over the acoustic windows of the ultrasonic probe as shown in FIG. 3B. In this embodiment, it is contemplated that the hydrogel be affixed to the probe 12 using any suitable method. In one embodiment, the hydrogel is attached to the probe with a cyanoacrylate-based adhesive such as ethyl cyanoacrylate (Loctite 406, Henkel) or octyl cyanoacrylate (Dermabond, Ethicon) dispersed in 2,2,4-trimethylpentane (Sigma-Aldrich, 360066), 1-octadecene (Sigma-Aldrich, O806), or paraffin oil (Sigma-Aldrich 18512) (See Wirthl et al., Instant tough bonding of hydrogels for soft machines and electronics, Science Advances, Vol. 3, No. 6 (2017).

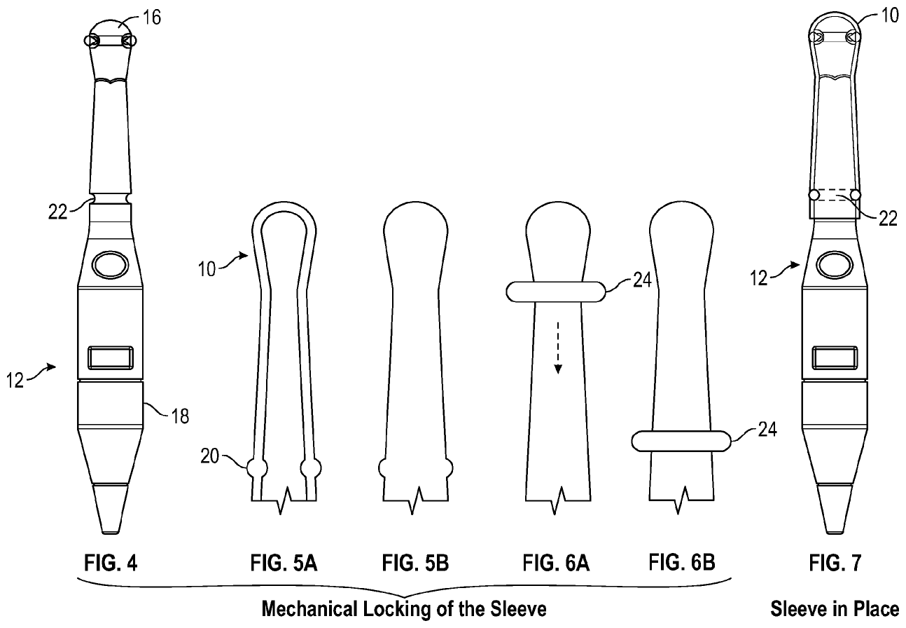

Referring again to the embodiment using the sleeve 10, the attachment of the sleeve 10 to the probe may be accomplished using a locking system. Referring now to FIG. 4, the probe 12 may include a distal imaging portion 16 and a proximal handle 18. In one embodiment, the locking system includes a sleeve 10 that is designed to be friction fit over the outer surface of the distal imaging portion 16 of the probe as shown in FIG. 5. In general, the sleeve material is elastic and can be pulled over the distal portion 16 of the probe 12 in order to attach, or lock, the sleeve 10 to the probe 12 by friction or compression forces. As shown in FIG. 5, the sleeve 10 may include an integral protuberance 20 configured to fit within and correspond to a circumferential recess 22 within the distal imaging portion 16 of the probe 12, further locking or affixing the sleeve 10 to the outer surface of the probe 12 as shown in FIG. 7.

In another embodiment, the locking system may be independent from the sleeve 10 and probe 12, such as a ring 24 adjusted to block the sleeve on top of the probe (FIG. 6A) or mechanically lock the sleeve 10 to the probe 12 to hold the sleeve in place during the exam (as shown in FIG. 6B). The ring 24 may be made from any suitable material.

The hydrogel couplant material may be designed to be compatible with the material of the construction of the sleeve 10. The appropriate attachment method may be chosen to suit the materials used to construct the hydrogel couplant material and the sleeve 10, and the preferred formulation of the hydrogel couplant material, i.e. whether the material encompasses the entire surface of the sleeve 10 or just a portion the surface of the sleeve 10 such as an imaging window (not shown).

The physical properties relevant to the performance of the hydrogel couplant material may be measured by numerous methods, including the procedures defined in Yi, J., Nguyen, et al., Polyacrylamide/Alginate Double-network Tough Hydrogels for Intraoral Ultrasound Imaging, Journal of Colloid and Interface Science, 578, 598-607 (2020), which is incorporated herein by reference. According to some embodiments, these designed properties of the hydrogel couplant include the initial water percentage (WP in the range of 50-90%) in the hydrogel after synthesis, which is measured by calculating the percent change in weight between initial sample and the weight of sample after vacuum drying for at least 144 hours at room temperature:

$$( WP = ( {Weight}_{initia1} - {Weight}_{d r y} ) / {Weight}_{initia1} ) ,$$

the equilibrium swelling ratio (ESR in the range of 50-90%), which is measured by calculating the percent change in weight of the vacuum dried sample and the weight of the rehydrated sample obtained by placing the vacuum dried sample in water for at least 24 hours, at room temperature:

$$( ESR = {Weight}_{hydration} - {Weight}_{dehydration} ) / {Weight}_{hydration} ) ,$$

stability in water (Rₜ in the range of 1±0.5), which is measured by calculating the ratio of the weight of the initial sample and the weight of the sample place in water for at room temperature after 24 hours:

$$( {R}_{t} = {W}_{24 hrs} / {W}_{i nitial} ) ,$$

toughness, i.e. the ability of a hydrogel to resist breaking when force is applied in the range of 0.1-100.0 MJ/m³:

$$U = {\int }_{0}^{ε failure} Sd ε$$

fracture energy of the hydrogel, i.e. the energy required to fracture the hydrogel in the range of 2.5-250 J/m²:

$$G = \frac{Ftear}{w}$$

the coefficient of friction of the hydrogel, i.e. the resistance to its ability to slide across the gingiva surface in the range of 0.1-10 for a normal force>2.5 mN:

$$μ \frac{f}{Fnormal}$$

an acoustic impedance (Z) in the range of 1.5±0.0.5 Mrays at 25 MHz:

Z=ρv,

where ρ is the density of the medium (in kg/m3) and v is the speed of sound through the medium (in m/s); and an acoustic amplitude attenuation<0.1 dB/mm/MHz and <2 dB/mm at 24 MHz,

$$A = {A}_{0} {e}^{- α z} ,$$

where A₀ is the unattenuated amplitude of the propagating wave at some location, A is the reduced amplitude after the wave has traveled a distance z from that initial location, the quantity α is the attenuation coefficient of the wave traveling in the z-direction (measured in nepers/length, where a neper is a dimensionless quantity), and e is the exponential (or Napier's constant) which is equal to approximately 2.71828.

According to some embodiments, the hydrogel couplant material is configured to include: WP in the range of 50-90%, ESR in the range of 50-90%. According to some other embodiments, the hydrogel couplant material is configured to comprise: a toughness in the range of 0.1-100.0 MJ/m3; a fracture energy in the range of 2.5-250 J/m2; a coefficient of friction of friction in the range of ˜0.1-10 for a normal force>2.5 mN. According to some other embodiments, an acoustic impedance in the range of 1.5+0.5 Mrays at 25 MHz; and an acoustic amplitude attenuation<0.1 dB/mm/MHz and <2 dBb/mm at 24 MHz. The relevant ultrasound properties are acoustic impedance and amplitude attenuation coefficient and may be calculated using known methods and procedures.

The hydrogel couplant material may be made from a variety of formulations and may include various monomers, initiators, polymers, surfactants, rheology modifiers, softeners, slip agents, anti-blocking agents, antiviral agents, antibacterial agents, flavorants, food grade colorants, and other materials or substances. Particular attention may be paid to achieving the desired physical properties of the formulation, such as viscosity and rheology, so as to satisfy the requirements of the processes (e.g., flow coating, dip coating, spray coating, etc. for two dimension films, injection molding, 3D printing, etc. to create three dimensional objects) required to fabricate the hydrogel couplant material that can be used to form the sleeve.

The choice of the processes used to fabricate the final sleeve 10 will depend on the choice of polymeric materials; e.g., whether it is a thermoset polymer or a thermoplastic polymer or a combination of the two, and the specific of class of thermoset or thermoplastic, whether it is homopolymer of heteropolymer, etc. It should be understood that the rheology and the viscosity requirements for each of the processes will vary depending on these choices.

It should also be understood that the hydrogel couplant material may include a composite of multiple (two or more) polymers and numerous additives. The additives may include surfactants, anti-blocking agents, anti-foammants, monomers, crosslinkers, initiators, chain propagation agents, softeners, antibacterial agents, antiviral agents, flavorants, food grade colorants, etc. And, the polymers may include natural polymers, such as alginates and gelatin, and synthetic polymers, such as polyacrylic acid, polyacrylamide, and polyethylene glycol. In one embodiment, the formulation and the process of fabrication are interdependent and are co-designed and co-optimized. In order to achieve the desired final properties, the fabrication process includes precise and accurate control of the process parameters, such as temperature, flow rate, rate heating and cooling, etc.

Examples of known hydrogel materials that may be suitable for use as the hydrogel couplant material includes, but are not limited to those found in Bahram, M., et al., An Introduction to Hydrogels and Some Recent Applications, Emerging Concepts in Analysis and Applications of Hydrogels (2016) (pH sensitive hydrogels such as those made from poly(acrylamide), poly(acrylic acid), poly(methacrylic acid, poly(diethylaminoethyl methacrylate), and poly(dimethylaminoethyl methacrylate); temperature sensitive hydrogels such as those made from poly(N-isopropylacrylamide) and poly(N,N-diethylacrylamide), collagen, agarose, hyaluronic acid, poly(organophosphazenes), and chitosan; electro-sensitive hydrogels such as those made from acrylamide and carboxylic acid derivatives; light-responsive hydrogels); Thompson, B. R., et al., An Ultra-Melt-Resistant Hydrogel from Food Grade Carbohydrates. RSC Advances, 7(72), 45535-45544 (2017) (such as those made from a binary hydrogel system of food grade biopolymers, such as agar and methylcellulose); Liu, J., et al., Functional Hydrogel Coatings, National Science Review, 8(2), nwaa254 (2021) (such as those made from hyaluronan and poly-D,L-Lactide, gelatin, PEG-heparin, PMBV/PVA, PHEMA, PEGPLA, PEGDA-co-AA, PEGDA, PHEMA, Chitosan/PVA, PAAm, PEG, PEG/polycarbonate, zwitterionic PCB, HEMA-co-DHPMA, pCBAA, PEGMA, PVA/PAA, PU, alginate/PPY, alginate/PEDOT, PVA/PEDOT, for example); Trzaskowski, M., et al., Hydrogel Coatings for Artificial Heart Implants, Challenges of Modern Technology, 2(1), 19-22 (2011) (polyvinylpyrrolidone (PVP) hydrogel); Yao, X., et al., Hydrogel Paint, Advanced Materials, 31(39), 1903062 (2019) (such as those made from covalently crosslinked polyacrylamide); Chimene, D., et al., Advanced Bioinks for 3D Printing: A Materials Science Perspective, Annals of Biomedical Engineering, 44(6), 2090-2102 (2016) (such as those made from methacrylated hyaluronan and poly(N-isopropylactrylamide) grafted hyaluronan, among others); Malda, J., et al., 25th anniversary article: Engineering Hydrogels for Biofabrication, Advanced Materials, 25(36), 5011-5028 (2013) (such as those made from alginate, collagen type 1, fibrinogen, poly(ethylene glycol), dimethacrylate, agar, agarose, fibrin, gelatin, atelocollagen, gelatin methacrylamide, chitosan, hyaluronan, hyaluronic acid, gellan, hydroxyethyl-methacrylate-derivatized-dextran, dyaluronic acid methacrylate, lutrol F127, lutrol, Matrigel, methylcellulose, N-isopropylamid, polyethylene glycol, poly(ethylene glycol) diacrylate, p(HPMAm-lactate)-PEG, and tetraPAc); Zhu, K., et al., A General Strategy for Extrusion Bioprinting of Bio-Macromolecular Bioinks through Alginate-Templated Dual-Stage Crosslinking, Macromolecular Bioscience, 18(9), 1800127 (2018) (gelatin methacryloyl hydrogel); Hong, S., et al., 3D Printing of Highly Stretchable and Tough Hydrogels into Complex, Cellularized Structures, Advanced Materials, 27(27), 4035-4040 (2015) (alginate-poly(ethylene glycol) hydrogel); McNulty, J. D., et al., Micro-Injection Molded, Poly (vinyl alcohol)-Calcium Salt Templates for Precise Customization of 3D Hydrogel Internal Architecture, Acta Biomaterialia, 95, 258-268 (2019) (polyacrylamide, polyethylene glycol-norbornene, and alginate hydrogels formed using poly(vinyl alcohol)-calcium slat templates); Negrini, N. C., et al., Three-Dimensional Printing of Chemically Crosslinked Gelatin Hydrogels for Adipose Tissue Engineering, Biofabrication, 12(2), 025001 (2020); and He, Y., et al., Research on the Printability of Hydrogels in 3D Bioprinting, Scientific Reports, 6, 29977 (2016) (3D hydrogel structures), all of which are incorporated herein by reference.

### EXAMPLE

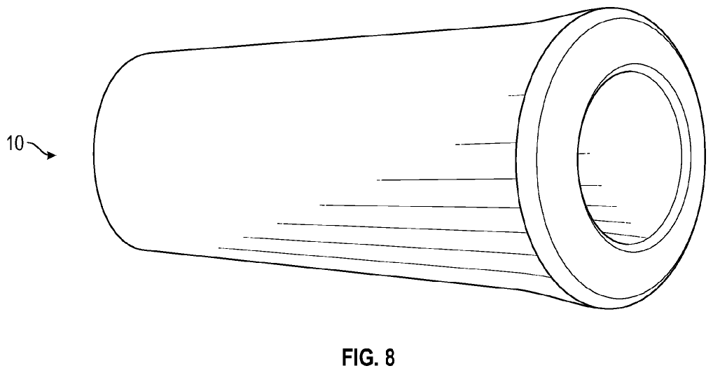

As shown in FIG. 8, a hydrogel couplant polymer material was formed using the following method. The following materials were acquired from Millipore Sigma: Low and medium viscosity sodium alginate, phosphate buffered saline tablets, acrylamide, N,N′-methylenebisacrylamide, N,N,N′,N′-tetramethylenediamine, ammonium persulfate, and calcium chloride.

A phosphate buffered saline tablet was dissolved in 200 ml of water, followed by an amount of sodium alginate sufficient to form a solution with sodium alginate concentration in the range of 0.25% to 5%. After the sodium alginate solution was equilibrated at room temperature for about 12-24 hours, 12 g of acrylamide, 0.08 g N,N′-methylenebisacrylamide, 90 micro liters of N,N,N′,N′-tetramethylenediamine, and 0.03 g of ammonium persulfate were added and the sodium alginate solution and mixed for another half hour.

The solution was then transferred to a test tube, into which a solid Teflon rod is inserted. The entire apparatus was then immersed in a water bath that was maintained at 50° C. for four hours. After four hours, the Teflon rod was removed from the test tube and the hollow polymerized sleeve was transferred from the test tube to a glass jar containing a 3 M solution of calcium chloride for 24 hours, after which the elastic polymer sleeve was removed from the calcium chloride solution and washed with deionized water and stored in a controlled environment, such as an enclosed environment or an open environment where the humidity is controlled.

This written description sets for the best mode of carrying out the invention and describes the invention so as to enable a person of ordinary skill in the art to make and use the invention by presenting examples of the elements recited in the claims. The detailed descriptions of those elements do not impose limitations that are not recited in the claims, either literally or under the doctrine of equivalents.

What is claimed is:

1. A hydrogel couplant system for use in ultrasonic imaging, the couplant system comprising:
a sleeve body comprising at least a hydrogel couplant material;
wherein the sleeve body is configured to at least partially encapsulate an ultrasonic probe; and
wherein the hydrogel couplant material is configured to cover an acoustic window of the ultrasonic probe.

2. The hydrogel couplant system of claim 1, wherein the system is configured to be used for ultrasonic intraoral imaging.

3. The hydrogel couplant system of claim 1, wherein the system is configured to be used for ultrasonic periodontal imaging.

4. The hydrogel couplant system of claim 1, wherein the hydrogel couplant material is a multicomponent composite hydrogel material.

5. The hydrogel couplant system of claim 1, wherein the ultrasonic probe comprises at least one transducer assembly.

6. The hydrogel couplant system of claim 5, wherein the transducer assembly comprises at least one transducer.

7. The hydrogel couplant system of claim 1, wherein the hydrogel couplant material has an initial water percentage in the range of about 50% to about 90%; an equilibrium swelling ratio in the range of about 50 to about 90%; a stability in water in the range of about 1±0.5; a toughness in the range of about 0.1 to about 100.0 MJ/m³; a fracture energy in the range of about 2.5 to about 250 J/m²; a coefficient of friction in the range of about 0.1 to about 10, for a normal force greater than 2.5 Mn; an acoustic impedance in the range of about 1.5±0.2 Mrays at 25 MHz;
and, an acoustic amplitude attenuation in the range of less than about 0.9 db/mm.

8. The hydrogel couplant system of claim 6, wherein the sleeve body is hypoallergenic.

9. The hydrogel couplant system of claim 7, wherein the sleeve body is antiviral.

10. The hydrogel couplant system of claim 7, wherein the sleeve body is antibacterial.

11. The hydrogel couplant system of claim 1, wherein the hydrogel couplant material is configured to comprise an initial water percentage in the range of about 50% to about 90%, an equilibrium swelling ratio in the range of about 50% to about 90%, and a stability in water in the range of about 1±0.5.

12. The hydrogel couplant system of claim 1, wherein the hydrogel couplant material is configured to comprise a toughness in the range of about 0.1 to about 100.0 MJ/m³; a fracture energy in the range of about 2.5 to about 250 J/m²; and, a coefficient of friction of friction in the range of about 0.1 to about 10 for a normal force greater than 2.5 Mn.

13. The hydrogel couplant system of claim 1, wherein the hydrogel couplant material is configured to comprise an acoustic impedance in the range of about 1.5±0.2 Mrays at 25 MHZ, and an acoustic amplitude attenuation in the range of less than 0.9 db/mm.

14. The hydrogel couplant system of claim 1, wherein the sleeve body contains at least one additive selected from the group comprising: surfactants, anti-blocking agents, anti-foammants, monomers, crosslinkers, initiators, chain propagation agents, softeners, antibacterial agents, antiviral agents, flavorants, and food grade colorants.

15. The hydrogel couplant system of claim 1, wherein the sleeve body further comprises at least one natural or synthetic polymer.

16. The hydrogel couplant system of claim 1, wherein the hydrogel couplant material is integrally formed within the body of the sleeve.

17. The hydrogel couplant system of claim 1, wherein the hydrogel couplant material is adhered to an outer surface of the sleeve body.

18. The hydrogel couplant system of claim 17, wherein the hydrogel couplant material is configured to cover substantially all of the outer surface of the sleeve body.

19. The hydrogel couplant system of claim 17, wherein the hydrogel couplant material is configured to cover a portion of the outer surface of the sleeve body.

20. A hydrogel couplant system for use in ultrasonic imaging, the couplant system comprising:
a hydrogel couplant material; and
an ultrasonic probe;
wherein the hydrogel couplant material is configured to cover at least a portion of an acoustic window of the ultrasonic probe.
